(19) United States
(12) Patent Application Publication
(10) Pub. No.: US 2025/0296229 A1
(43) Pub. Date: Sep. 25, 2025

(54) SYSTEMS AND METHODS FOR MAKING BIOLOGICAL ROBOTS

(71) Applicants: Trustees of Tufts College, Medford, MA (US); University of Vermont, Burlington, VT (US)

(72) Inventors: Michael Levin, Medford, MA (US); Joshua Clifford Bongard, Burlington, VT (US)

(21) Appl. No.: 18/869,701

(22) PCT Filed: May 26, 2023
(86) PCT No.: PCT/US2023/067545

§ 371 Date: Nov. 26, 2024

Related U.S. Application Data
(60) Provisional application No. 63/346,142, filed on May 26, 2022.

(51) Int. Cl. B25J 9/16

(52) U.S. Cl. CPC B25J 9/163

(57) ABSTRACT

Various systems and methods for making biological robots are disclosed. An example system can include a well plate with a receptacle, a robotic assembly configured to form a biological robot in the receptacle by dispensing biological material into the receptacle, an insert positioned to interact with the receptacle including one or more components configured to stimulate to the biological robot, and a camera system configured to monitor the biological robot in the receptacle. The example system can further include a computing system configured to receive an input from a user indicative of a desired behavior of the biological robot, apply the user input as input to an artificial intelligence model, identify a stimulus for providing to the biological robot, and cause the insert to provide the stimulus to the biological robot.

### CROSS-REFERENCE TO RELATED APPLICATIONS

This application claims the benefit of and priority to U.S. Provisional Patent Application No. 63/346,142, filed May 26, 2022, the entirety of which is incorporated by reference herein.

### STATEMENT REGARDING FEDERALLY SPONSORED RESEARCH

This invention was made with government support under grant 2020247 awarded by the National Science Foundation. The government has certain rights in the invention.

### TECHNICAL FIELD

The technical field generally relates to engineered biological robots (“biobots”), and systems and methods for making biological robots. Generally, biological robots are synthetic lifeforms that are designed using computers to perform a desired function, and they are built using different types of cells. There are different types of biological robots, and biological robots can be described using a variety of language and terminology.

### BRIEF DESCRIPTION

One aspect of the present disclosure is a system for making biological robots. The system includes a well plate including a receptacle; a robotic assembly configured to form a biological robot in the receptacle by dispensing biological material into the receptacle; an insert positioned to interact with the receptacle and including one or more components configured to stimulate to the biological robot; a camera system configured to monitor the biological robot in the receptacle; and a computing system communicatively coupled to the robotic assembly and the camera system. The computing system is configured to receive an input from a user indicative of a desired behavior of the biological robot; apply the user input as input to an artificial intelligence model; identify a stimulus for providing to the biological robot such that the biological robot exhibits the desired behavior based on an output of the artificial intelligence model; and cause the insert to provide the stimulus to the biological robot.

Another aspect of the present disclosure is a method for making biological robots. The method includes maintaining, by a computing system, an artificial intelligence model used for designing biological robots; generating, by the computing system based on a first output of the artificial intelligence model, a first design for a biological robot; causing, by the computing system, a robotic assembly to form the biological robot based on the first design in a first contained environment; causing, by the computing system, a stimulus to be provided to the biological robot in the first contained environment; receiving, by the computing system, data indicative of a response that is exhibited by the biological robot to the stimulus; training, by the computing system, the artificial intelligence model using the data indicative of the response that is exhibited by the biological robot to the stimulus; generating, by the by the computing system based on a second output of the artificial intelligence model, a second design for the biological robot; and causing, by the computing system, the robotic assembly to form the biological robot based on the second design in a second contained environment.

Yet another aspect of the present disclosure is another system for making biological robots. The system includes a well plate including a plurality of receptacles; a motion system configured to control a position of a head that that dispenses biological material into the plurality of receptacles to form biological robots in the plurality of receptacles; an insert positioned to interact with the plurality of receptacles, the insert comprising one or more components configured to provide a stimulus to the biological robots; a camera system disposed over the plurality of receptacles; and a controller communicatively coupled to the insert, the camera system, and the motion system, the controller configured to cause the insert to provide the stimulus to the biological robots based on instruction from a computing system and to provide data from the camera system to the computing system.

### BRIEF DESCRIPTION OF THE FIGURES

FIG. 1 is an illustration of an example process for making biological robots, in accordance with some aspects of the disclosure.

FIG. 2 is a diagram illustrating an example system for making biological robots, in accordance with some aspects of the disclosure.

FIG. 3 a block diagram showing an example architecture for the system of FIG. 2, in accordance with some aspects of the disclosure.

FIG. 4 is an illustration of an example system for housing biological robots, in accordance with some aspects of the disclosure.

FIG. 5 is an illustration of another example system for housing biological robots, in accordance with some aspects of the disclosure.

FIG. 6 is a drawing showing example software functionality associated with different components of the system of FIG. 2, in accordance with some aspects of the disclosure.

FIG. 7 is a diagram illustrating an example process for making xenobots using the system of FIG. 2, in accordance with some aspects of the disclosure.

FIG. 8A shows a diagram illustrating an example process for making anthrobots using the system of FIG. 2, in accordance with some aspects of the disclosure.

FIG. 8B is a continuation of FIG. 8A.

FIG. 9 is a block diagram showing example layers of the system of FIG. 2, in accordance with some aspects of the disclosure.

FIG. 10A is an illustration of an example biological robot receptacle tray, in accordance with some aspects of the disclosure.

FIG. 10B is a close-up view of a portion of the illustration of FIG. 10A.

FIG. 11 is an illustration of an example xenobot receptacle control system, in accordance with some aspects of the disclosure.

FIG. 12 is an illustration of an example anthrobot receptacle control system, in accordance with some aspects of the disclosure.

FIG. 13 is an illustration of an example biological robot receptacle control system, in accordance with some aspects of the disclosure.

FIG. 14A is an illustration of a monitoring system for biological robots, in accordance with some aspects of the disclosure.

FIG. 14B is another view of the illustration of FIG. 14B.

FIG. 15 is an illustration of an example process for designing and making biological robots, in accordance with some aspects of the disclosure

### DETAILED DESCRIPTION

The following discussion is presented to enable a person skilled in the art to make and use aspects of the disclosure. Various modifications to the illustrated aspects will be readily apparent to those skilled in the art, and the principles discussed herein can be applied to other applications without departing from aspects of this disclosure. The following detailed description is to be read with reference to the figures. The figures, which are not necessarily to scale, depict selected aspects of the disclosure and are not intended to limit the scope of the disclosure. Skilled artisans will recognize the examples provided herein have many useful alternatives that fall within the scope of the disclosure.

Biological robots are synthetic lifeforms that are designed using computers (in silico) to perform a desired function, and they are built using different types of cells. Artificial intelligence can be used in the design of biological robots to dynamically discover and create lifeforms to serve an intended purpose. Some biological robots can be very small in size, such as less than 1 millimeter wide. Biological robots can include different types of cells, such as skin cells and heart muscle cells (in addition to any other kind of cells). The skin cells can act as structural support (in addition to sensing chemicals, pressure, temperature, light, electric fields, etc. and doing computations), where the heart cells can act as “motors” (in addition to other functions). The cells used to build biological robots can be derived from stem cells, such as harvested from early (blastula stage) frog embryos, as well as human cells. Biological robots created from frog cells have been referred to as “xenobots”, whereas biological robots created from human cells have been referred to as “anthrobots”.

It will be appreciated that biological robots may not necessarily be accepted as “robots” yet, but over time may become accepted as some type of new organism, another type of life form entirely, or may be referred to more generally using terms such as “engineered organisms”, “reconfigurable organisms”, or the like. They are robots in the sense that their structure and function can be rationally controlled, and the system can learn ways to program the self-assembly of cells into functional biobots, gastruloids, organoids, or the like.

The body shape of a given biological robot and the distribution of different types of cells (e.g., skin and heart cells) can be automatically designed using computer simulations so the biological robot can perform a specific task. The process of designing biological robots can include trial and error, such as through use of various types of evolutionary algorithms. Biological robots can perform different tasks such as walking, swimming, pushing, carrying, and working together in a swarm. In some cases, biological robots can survive for weeks without food and can heal themselves after lacerations.

Different types of cells and other structures can be incorporated into biological robots to act as motors and cells. For example, instead of heart muscle, some biological robots can grow patches of cilia and use them as “oars” for swimming or otherwise moving. Moreover, deoxyribonucleic acid (DNA) or ribonucleic acid (RNA) molecules can be introduced to give biological robots a sense of memory, or implement any of a myriad available synthetic biology circuits which perform new metabolic, computational, or behavioral functions. For example, if exposed to a certain kind of light, the biological robot might glow a predetermined color when viewed under a fluorescence microscope. Biological robots can also self-replicate by gathering loose cells from their environment and forming them into new biological robots with similar capabilities.

Biological robots can potentially be used for a variety of different applications. They can be used as a scientific tool to further understand how cells cooperate to build complex bodies, such as during morphogenesis. They are also biodegradable and biocompatible, which can lead to a variety of different uses. In a controlled setting such as in a Petri dish (in vitro), swarms of biological robots can work together to push microscopic pellets into central piles. In a similar fashion, biological robots could be used to aggregate microplastics in the ocean into a large ball that a drone or a boat could gather and bring to a recycling center, for example. Biological robots do not add pollution because they simply work and degrade by using energy from fat and protein. Then, when biological robots run out of energy, they turn into dead cells that are biodegradable. Biological robots could also be used for drug delivery in the sense that they could be made from the cells of a human patient and bypass immune response challenges presented by other kinds of micro-robotic delivery systems. Similar biological robots could also be used to scrape plaque from arteries, locate and treat different types of diseases, and a variety of other possible applications.

Additional detail regarding biological robots (engineered multicellular organisms) can be found in PCT Patent Application Nos. US2021/013105, filed Jan. 12, 2021, US2021/061222, filed Nov. 30, 2021, and U.S. patent application Ser. No. 17/647,847, field Jan. 12, 2022, the entire contents of each of which is incorporated by reference herein.

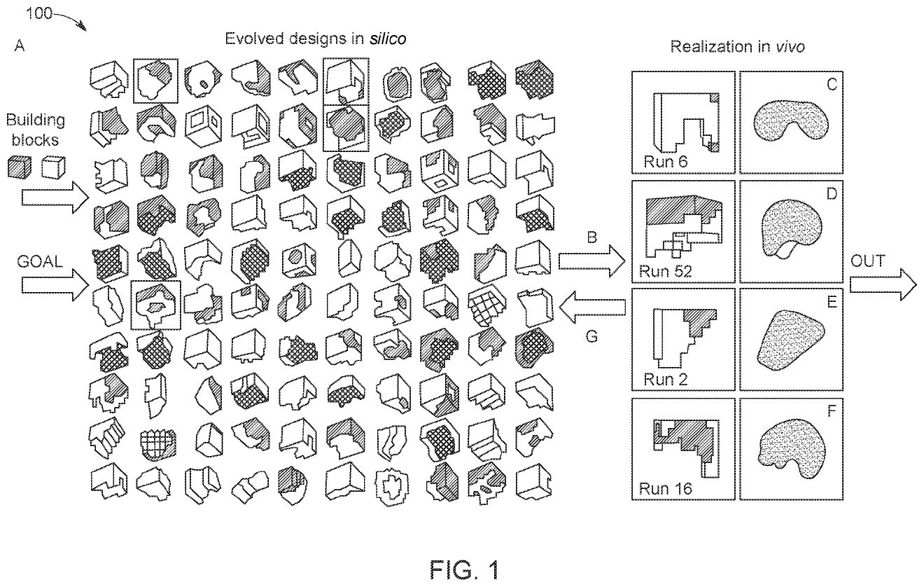

Referring to FIG. 1, an illustration of an example process 100 for making biological robots is shown, in accordance with some aspects of the disclosure. The process 100 as illustrated in FIG. 1 shows a general overview of the functionality performed by the system 200 described in detail below. The process 100 uses certain structural building blocks (e.g., different types of cells, tissues, etc.) and a desired behavioral goal as input to an evolutionary algorithm. The evolutionary algorithm then evolves an initially random population and returns the best biological robot design that was discovered. The algorithm can then rerun (e.g., 99 times) starting with different random populations, thereby generating a diversity of performant designs in silico. The performant designs can then be filtered by robustness to random phase modulation of contractile cells, constructed in vivo using developing Xenopus cardiomyocyte and epidermal cell progenitors, and placed on the surface of a Petri dish where their behavior can be observed and compared to the predicted behavior of the design. Any discrepancies between in silico and in vivo behavior can be returned to the evolutionary algorithm in the form of constraints on the kind of designs that can evolve during subsequent design-manufacture cycles. Concurrently, different tissue layering and shaping techniques can be modified such that realized living systems behave more like their virtual models.

To facilitate more widespread usage of biological robots, practical systems and methods for making biological robots are needed. Major progress in the biomedicine of birth defects, cancer, regenerative repair, and synthetic bioengineering depend on the ability to stimulate cells to build desired structures (in vivo or in vitro). The goal of learning how to control cells toward specific anatomical outcomes is stymied by the slow progress of manual experiments in synthetic and developmental biology. The cycle of “hypothesize, let cells build, evaluate outcome, revise hypothesis” is currently being done by human scientists and is inherently difficult, expensive, and time consuming.

The systems and methods of the present disclosure involve the design of an automated high-throughput platform (e.g., the system 200). The automated platform may function as a “discovery engine” or a “robot scientist,” in which machine learning guides a machine that stimulates cells to build biological robots. The machine can evaluate the structure and behavior of the biological robots, and revise hypotheses about the morphogenetic code, to be tested in the next cycle. This cycle repeats, in parallel, enabling the machine to refine its understanding of the mapping from stimuli given to cells (electrical, optical, chemical, biomechanical, etc.) to the resulting morphology. This discovery engine is expected to churn out bespoke synthetic living machines for useful purposes, in addition to fundamental new knowledge about control of cell and tissue behavior. The discovery engine can learn to stimulate agential materials (cells—components with agendas) to exploit their internal complexity, programming living matter with signals (as we do with computing devices) not by micromanagement. The machine is different from robot scientist platforms for biomechanical experiments (such as automated screening for metabolic pathways, etc.) in single cells.

Some aspects of the system for multicellular morphology (e.g., the system 200) may include the use of machine learning to control and understand morphogenesis, the ability to provide specific novel synthetic living machines, and the ability to provide biological robots in bulk (high throughput) without human intervention, made from any kind of cell (human, xenopus, any other types of species). This technology can be useful for any area in which synthetic living machines, or stimuli for morphogenesis (regenerative medicine) are useful, and any area in which known biological robots need to be deployed at large scale (environment, industry, etc.). With the systems and methods provided herein, biological robots are no longer limited by the need for human scientists' construction. Moreover, the system doesn't just make one kind of biological robot, like standard automation technologies, but can make novel types of biological robots as needed and learn from each run so that its performance and capabilities improve with experience. In this way, the systems and methods may operate in accordance with multiple operating modes. For example, one mode may be referred to as “engineering mode”, where the systems and methods are designed to create a biological robot that, for example, performs a particular specified function or achieves a specified result. In another mode, the systems and methods may operate in a “discovery mode”, wherein the systems and methods create or find new biology with desired or specified attributes. Discovery mode may be particularly useful, for example, for applications in regenerative medicine or swarm robotics.

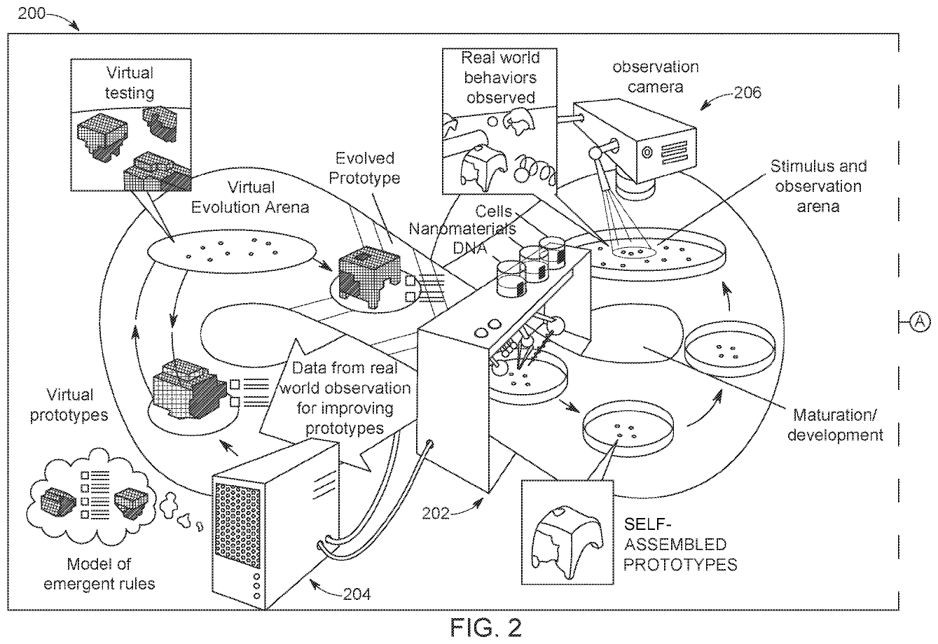

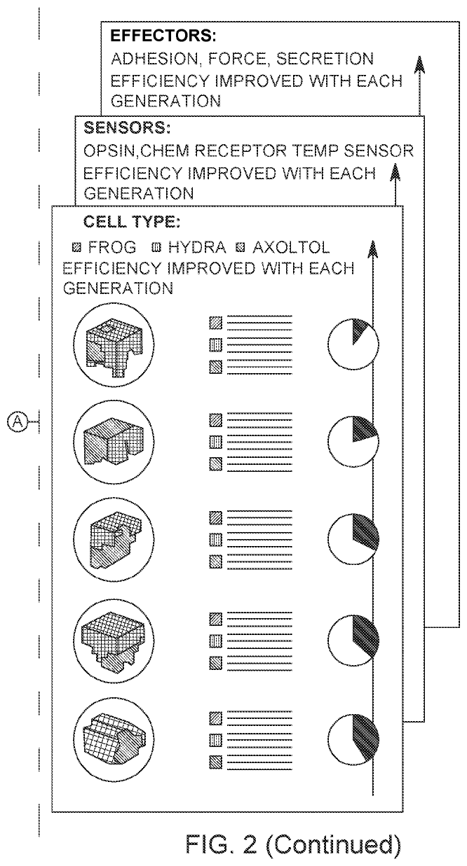

Referring to FIG. 2, a diagram illustrating an example system 200 for making biological robots is shown, in accordance with some aspects of the disclosure. The system 200 is generally a biomanufacturing system including one or more biomanufacturing devices. The system 200 includes one or more computing devices for maintaining and training one or more machine learnings models (among other functions), including one or more models of emergent rules that are developed through experimentation with real and virtual biological robots (e.g., the first model 1530 and the second model 1540 described below). The system 200, thus, may operate according to one or more operating modes. In some configurations, the system 200 may employ multiple machine learning modes and/or multiple instances of learning networks or “artificial intelligence” models. In one non-limiting example, multiple networks or artificial intelligences may be configured in opposition or in a “rival” configuration. The system 200 can be referred to as a “Mom Bot” system that has sufficient modularity and flexibility necessary to support biological robot research and production.

The system 200 can develop virtual prototypes of biological robots for a desired application, for example by using certain structural building blocks (e.g., different types of cells, tissues, etc.) and a desired behavioral goal as input to an evolutionary algorithm (e.g., the first model 1530 and the second model 1540 described below). The system 200 can then conduct virtual testing of the virtual biological robot prototypes in a virtual arena and evolve the virtual prototypes, for example using real world data form biological robot prototypes to train machine learning models (e.g., supervised learnings, unsupervised learning, reinforcement leaning, regression models, decision trees, K-means, random forests, different types of neural networks, and the like, such as the first model 1530 and the second model 1540 described below). The system 200 can also include different types of controllers for performing different functions within the system.

The system 200 is shown to include a robotic assembly 202 that physically produces biological robots, for example in a Petri dish. The robotic assembly can include one or more multi-function heads and/or pipettes that are moved between positions by one or more robotic motion systems to dispense and/or extract biological materials (e.g., into/from Petri dishes contained in receptacles), as discussed for example in further detail below. The robotic assembly 202 is shown to be in communication with a computing system 204 that receives the evolved biological prototype designs and implement them in the real world. The computing system 204 can also be in communication with (e.g., communicatively coupled to) the observation camera system 206. The computing system 204 can include one or more computing devices (e.g., the first control computer 220 and the second control computer 230 discussed below), including on-premises computing devices and/or remote (cloud-based) computing devices. The robotic assembly 202 can be implemented using a variety of different types of hardware and software configurations, and can include components such as pivoting arms and dispensers used to produce biological robots as well as conduct different experiments with the biological robots. The robotic assembly 202 can use different types of biological materials including cells, nanomaterials, DNA, and other types of biological materials to produce the biological robots, for example. Different cell types such as frog cells, hydra cells, and axolotl cells can be used to create the biological robots. The efficiency of the system 200 can improve with each generation iteration.

Once the real-world prototypes have self-assembled after cells are placed in an environment by the robotic assembly 202, they can experience a process of maturation and development. For example, the automatically self-assembled prototypes can observe their environment (e.g., in a Petri dish) and interact with the environment in different manners. Different types of stimuli may be applied to the prototype biological robots by the system 200, and effectors such as adhesion, force, and secretion can be observed. The different types of stimuli affect how the biological robots self-assemble, and the system 200 can learn from the response of the biological robots to discover stimuli that make the cells create desired structures. Different types of sensors can be included and used in the system 200 to generate data such as opsin sensors, chemical receptor sensors, temperature sensors, and other types of sensors. The system 200 can further include an observation camera system 206 that can observe the behavior of the biological robot prototypes, including behavior in response to different stimuli that are provided. The data collected from observing the behavior of the biological robots can be used to train the one or more machine learning models (e.g., the first model 1530 and the second model 1540 described below) to improve efficiency of the process for making the biological robots performed by the system 200 with each successive generation.

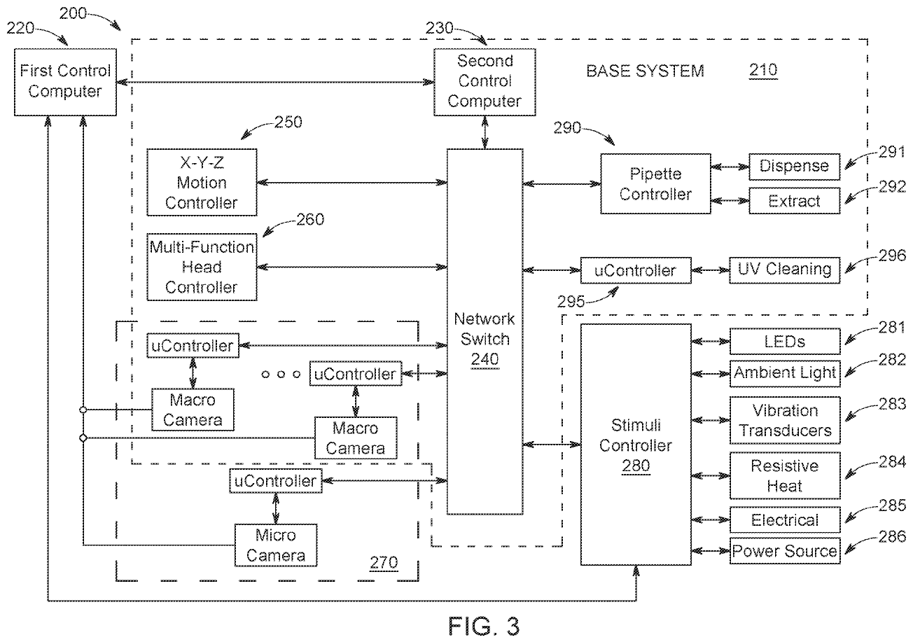

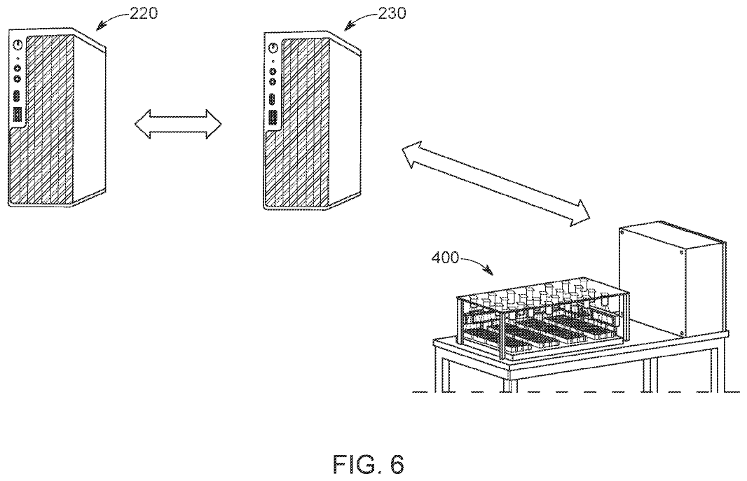

Referring to FIG. 3, a block diagram showing an example architecture for the system 200 is shown, in accordance with some aspects of the disclosure. The system 200 is shown to include a network switch 240 that is connected to a variety of different controllers. The network switch 240 can be implemented using a variety of different types of hardware and software configurations, and can also be implemented as either one or multiple network switches. A first control computer 220 is shown to be connected to the network switch 240, and a second control computer 230 is shown to be connected to the first control computer 220. The functions performed by the first control computer 220 and the second control computer 230 are discussed in more detail below with respect to FIG. 6. As shown in FIG. 3, the architecture of system 200 an include a base system 210 that includes different components as shown as well as components not included in the base system 210. It will be appreciated that a variety of different custom engineering solutions can be assembled to form the system 200, including system components produced and/or designed by different entities.

In FIG. 3, the network switch 240 is also shown to be connected to an x-y-z motion controller 250. The x-y-z motion controller 250 can be configured to operate the robotic assembly 202, for example, to control the position of a multi-function head for dispensing biological materials and removing biological materials. The x-y-z motion controller 250 can likewise be configured to control the position of the multi-function heads of the motion systems 410, 1110, 1210, and 1310 discussed below, for example. The network switch 240 is also shown to be connected to a multi-function head controller 260 that can be used to control operation of the multi-function head connected to the robotic assembly 202, in addition to other functionality. The network switch 240 is also shown to be connected to a variety of different microcontrollers, including a plurality of microcontrollers 270 and a microcontroller 295. The microcontrollers 270, as shown, can be used to operate different macro and micro level camera devices of the observation camera system 206. The microcontroller 295, as shown, can be used to control an ultraviolet cleaning device 296 of the system 200. The network switch 240 is also shown to be connected to a pipette controller 290, which can be configured to control dispensing actions 291 and extracting actions 292 of biological materials used to make and experiment with biological robots.

The network switch 240 is also shown to be connected to a stimuli controller 280, which can be configured to control a variety of different stimuli (interventions) provided to the self-assembled real-world biological robots made by the system 200. For example, as shown, the stimuli controller 280 can be configured to control operation of different sources of stimuli including light-emitting diodes (LEDs) 281, ambient lighting 282, vibration transducers 283, resistive heat 284, and other types of stimuli that can be provided to the prototype biological robots made by the system 200. The stimuli controller 280 can also include various electrical connections 285 to different devices that can provide stimuli, as well as being connected to a power source 286. In some examples, as noted, only certain components of the system 200 as illustrated in FIG. 3 are provided as part of the base system 210 offering, whereas other components are added to the base system depending on the intended application by the user.

The components of the system 200 can be implemented using a variety of different types of hardware and software configurations as appreciated by the skilled person. For example, the computing devices and controllers can be implemented in a variety of manners (e.g., one or more electrical circuits in a variety of configurations), and can generally include one or more processing devices (e.g., central processing units (CPUs), graphics processing units (GPUs), etc.), memory (e.g., volatile memory, non-volatile memory, storage, RAM, ROM, EEPROM, one or more flash drives, one or more hard disks, one or more solid state drives, one or more optical drives, non-transitory computer-readable storage media, etc.), communications interfaces (e.g., hardware, firmware, and/or software that can be used to establish an Ethernet connection, a USB connection, a Wi-Fi connection, a Bluetooth connection, a cellular connection, a coaxial connection, a fiber optic connection, etc.), and user interface elements (e.g., indicators, sensors, screens, actuatable buttons, a keyboard, a mouse, a graphical user interface, a touch-screen display, etc.). The one or more computing devices can be devices such as a personal computer, workstation, smartphone, tablet, one or more servers (such as either an on-premises server computer and/or a remote (cloud) server), and other types of devices and combinations thereof.

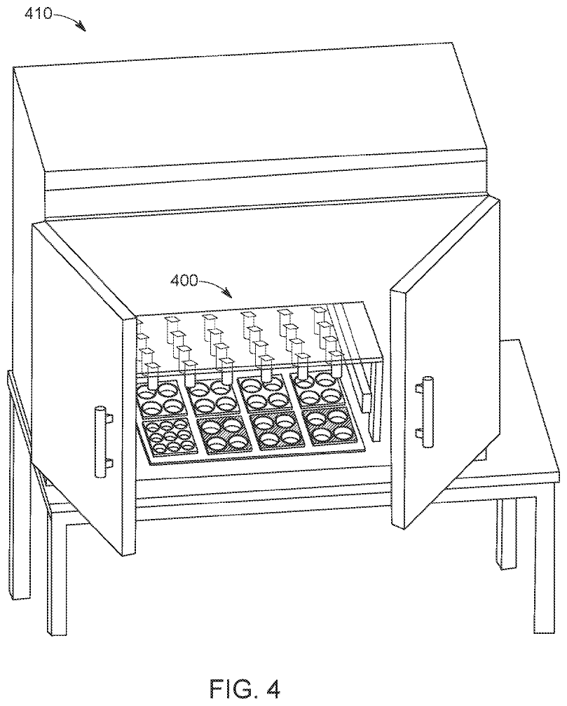

Referring to FIG. 4, an illustration of an example system for housing biological robots is shown, in accordance with some aspects of the disclosure. Once the prototype biological robots have been made by the system 200, they can be housed in a benchtop incubator 410 as illustrated in FIG. 4. The benchtop incubator 410 can maintain a consistent environment for the biological robots (e.g., anthrobots), and can also allow for temperature variation during the growth phase for biological robots made by the system 200 (e.g., xenobots). The lower layers (e.g., the well pates) of a monitoring system 400 for the biological robots (discussed in more detail below) can slide out of the benchtop incubator 410 for loading and unloading different biological robot protypes.

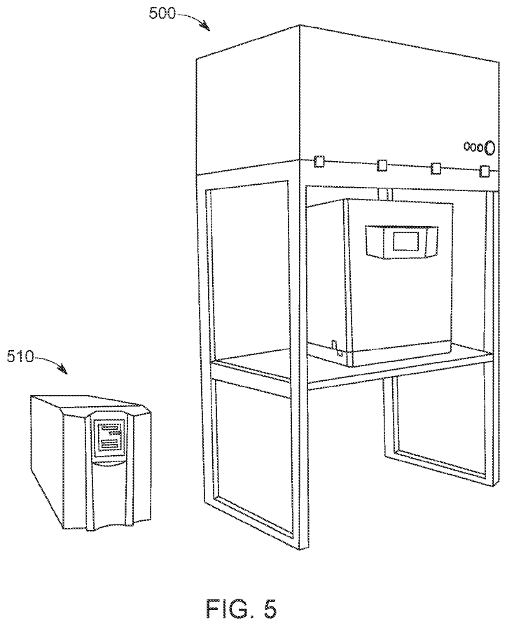

Referring to FIG. 5, an illustration of another example system for housing biological robots is shown, in accordance with some aspects of the disclosure. Specifically, a laminar flow hood 500 is shown, which can be used to maintain a sterile environment for any systems or objects maintained within the laminar flow hood. In some examples, the benchtop incubator 410 can be placed within the laminar flow hood 500. However, other types of systems and devices that house biological robots can also be placed within the laminar flow hood 500 to maintain a sterile environment. The laminar flow hood 500, the benchtop incubator 410, and/or associated components can be powered by a separate uninterruptable power supply 510 as shown in FIG. 5. The uninterruptable power supply 510 can be configured to hold power for the laminar flow hood 500 until a backup generator turns on, for example.

Referring to FIG. 6, a drawing showing example software functionality associated with different components of a system for making biological robots is shown, in accordance with some aspects of the disclosure. Specifically, FIG. 6 shows the first control computer 220 and the second control computer 230 as shown in FIG. 3 as well the monitoring system 400 as shown in FIG. 4. The first control computer 220 can be configured to describe available capabilities (e.g., capabilities of the robotic assembly 202, capabilities of the different controllers in FIG. 3, etc.) of the system 200 for making biological robots. The capabilities can include availability of different types of cells, nanomaterials, and DNA that can be used to make biological robots, for example, in addition to different types of monitoring capabilities, stimuli, etc. The first control computer 220 can also be configured to execute received commands, for example to control different hardware components of the system 200 and provide command responses and statuses. The first control computer 220 can also be configured format and deliver camera images (e.g., obtained by the observation camera system 206), as well as monitor and report data on health of equipment in the system 200. The camera images produced by the observation camera system 206 may include fluorescent or luminescent images. That is, the cameras in the observation camera system 206 may be configured to acquire fluorescent or luminescent data. The cameras in the observation camera system 206 may have a spatial resolution capable of resolving single cells. Additionally, confocal microscopy capabilities may be integrated with or supplement the in the observation camera system 206. The first control computer can be configured to provide command responses such as complete or error, system status such as configuration, go or no go, and camera images with specific metadata (e.g., camera ID, etc.).

The second control computer 230 can be configured to perform a variety of different functions, including planning of experiments conducted by the system. For example, the second control computer 230 can be configured to plan different activities and stimuli to be executed during growth, screening, and observation stages (as discussed in more detail below). The second control computer 230 can also be configured to setup and control cameras in the observation camera system 206 and deliver commands to execute the activities and stimuli delivery. The second control computer 230 can further be configured to perform image interpretation during all phases of experiments, including tagging and tracking of biological robots, and interpretation of behavior exhibited by biological robots. The second control computer 230 can additionally be configured to cue activities based on image interpretation, including selection of different types of biological robots and micro vision data collection. The second control computer 230 can further provide command regarding system setup (e.g., camera settings for the observation camera system 206), stimulus application (e.g., location, function, parameters, duration), biological robot relocation (e.g., start location, end location), asynchronous image capture, and other types of commands and parameters.

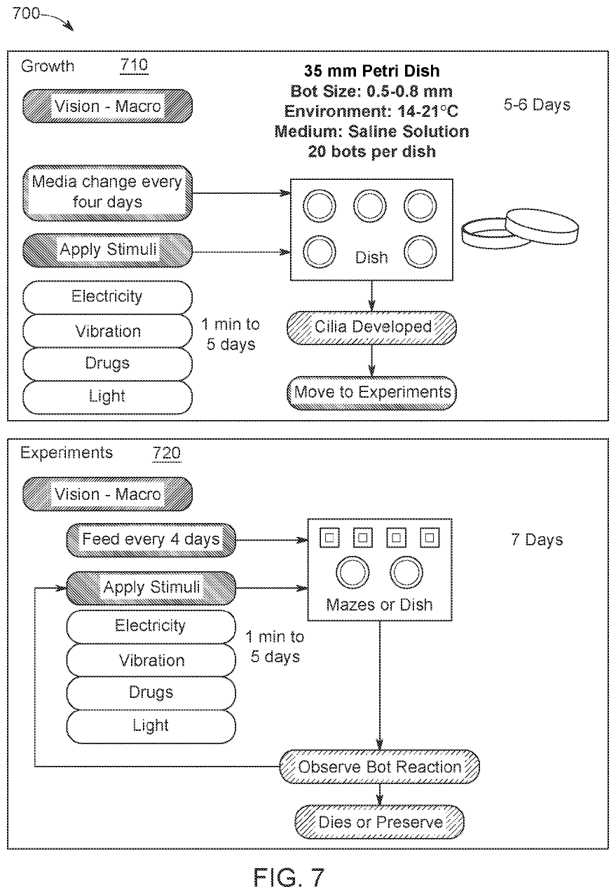

There are three major functions involved in making biological robots using the systems and methods discussed herein: growth, screening, and experiments. Referring to FIG. 7, a diagram illustrating an example process 700 for making xenobots is shown, in accordance with some aspects of the disclosure. The process 700 can be performed by the system 200 and includes both a growth phase 710 and an experiments phase 720. The growth phase 710 for xenobots can take 5-6 days and can take place in a 35 mm Petri dish, in some examples. The size of the xenobots, in some examples, can be 0.5-0.8 millimeters, the environment can be kept at 14-21° C., a saline solution medium can be provided within each Petri dish (and changed every four days, for example), and 20 biological robots can be placed in each Petri dish. Various types of stimuli can be applied during the growth phase, including electrical, vibration, light, and different types of drugs. The stimuli can be administered over a time period ranging from one minute to five days, in some examples. The xenobots can be observed using macro-level vision cameras during the growth phase 710, and the xenobots can develop cilia during the growth phase 710.

During the experiments phase 720, the xenobots can be fed every 4 days, for example. Moreover, one or more mazes can be inserted into each Petri dish, or the xenobots can be transferred from Petri dishes to mazes, to further study how the xenobots navigate their environment using the grown cilia. The experiments phase 720 can occur over a period of about 7 days, and the xenobots can be fed about every 4 days during the experiments phase, in some examples. Various types of stimuli can again be applied during the experiments phase 720, including electrical, vibration, light, and different types of drugs. The stimuli can again be administered over a time period ranging from one minute to five days, in some examples. After the stimuli are applied, the behavior of the xenobots can be observed, for example using macro-level vision cameras. If the behavior of the xenobots is desirable, the xenobots can be preserved, otherwise the xenobots can stop being fed such that they eventually die. Data regarding the behavior of the xenobots captured during the experiments phase 720 can be used to train machine learning models maintained by the system 200 (e.g., the first model 1530 and the second model 1540 described below). The process 700 can generally be repeated to automatically design and make xenobots with desirable characteristics.

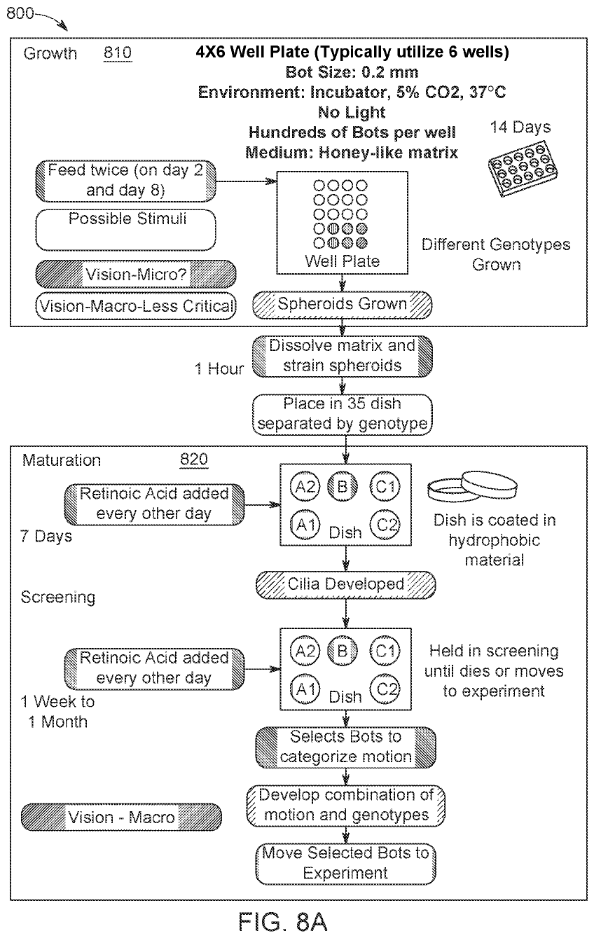

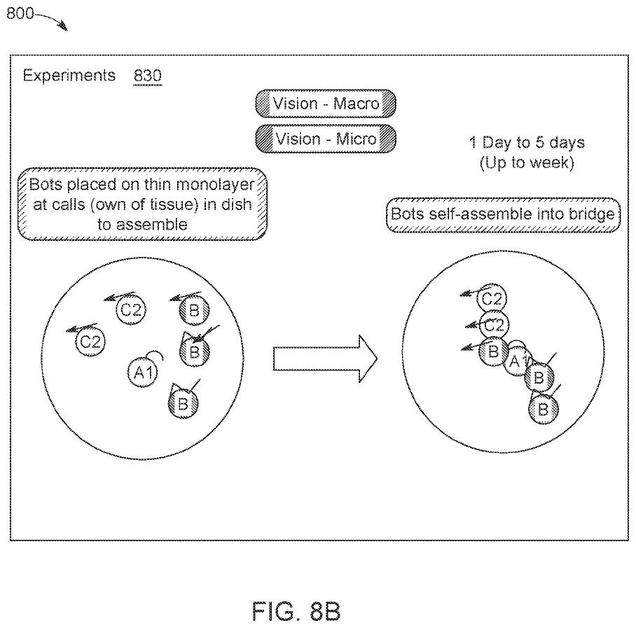

Referring to FIGS. 8A-8B, a diagram illustrating an example process 800 for making anthrobots is shown, in accordance with some aspects of the disclosure. The process 800 can be performed by the system 200, and is similar to the process 700 for making xenobots discussed above, however there are some notable differences. The process 800 for making xenobots, as shown, includes a growth phase 810, a maturation phase 820, and an experiments phase 830. The growth phase 810 for anthrobots can take place over 14 days and can take place in a 4×6 well plate (or other type of well plate), in some examples. The size of the anthrobots in some examples is about 0.2 millimeters wide, the environment can be within an incubator kept at about 37° C. where the atmosphere contains about 5% carbon dioxide (CO₂) and there is no light. In some examples, hundreds of anthrobots are formed in each well, and the medium within each well is a honeycomb-like matrix. The anthrobots can be observed during this growth phase 810 using micro-level vision cameras, macro-level vision cameras, or a combination of both. During the growth phase 810, the anthrobots are fed twice (on day 2 and day 8), in some examples, and stimuli may or may not be provided. Different genotypes can be gown during the growth phase 810 within different wells of the well plate, and at the end of the growth phase 810 the anthrobots can grow spheroids. Over a time period of about one hour, the anthrobots can be moved from the growth phase 810 to the maturation phase 820 by dissolving the matrix, straining the spheroids, and placing the anthrobots in Petri dishes (e.g., a 35-millimeter diameter Petri dish) that are coated in hydrophobic material and separated by genotype.

During the maturation phase 820, retinoic acid or another similar type of solution can be added to each Petri dish every other day for a period of about 7 days. As a result, most or all the anthrobots will develop cilia, enabling them to move about their environment. Then, the process transitions to a screening subphase of the maturation phase 820, where retinoic acid or another similar type of solution can be added to each Petri dish every other day for a period of about 1 week to 1 month, in some examples. The anthrobots can be held in the screening subphase until they die or move to the experiments phase 830. Anthrobots can be selected to move on to the experiments phase 830 to categorize motion or develop certain combinations of motion and genotypes. During the maturation phase 820 and the screening phase 830, the behavior of the anthrobots can be observed using macro-level vision cameras.

The anthrobots that are selected to move on to the experiments phase 830 can be placed on a thin monolayer of cells (lawn of tissue) in a Petri dish to assemble. Then, the anthrobots in the Petri dish can self-assemble into a bridge, as shown in FIG. 8B. The experiments phase 830 for the anthrobots can take 1 to 5 days, in some examples, and may take up to a week. The experiments phase 830 for the anthrobots can be observed using both macro-level and micro-level vision cameras, and data collected form observing the anthrobots can be used to train machine learning models maintained by the system 200 (e.g., the first model 1530 and the second model 1540 described below). The process 800 can generally be repeated to automatically design and make anthrobots with desirable characteristics.

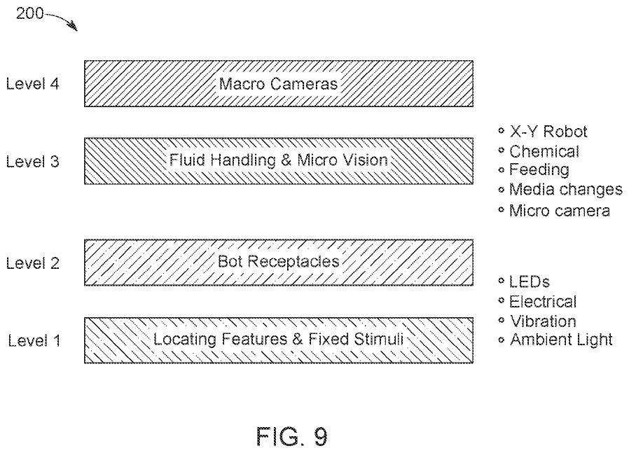

Referring to FIG. 9, a block diagram showing example layers of the system 200 is shown, in accordance with some aspects of the disclosure. The different layers illustrated in FIG. 9 can be physical/functional subsystem layers/levels of the system 200 that interact with biological robots. The system 200 can be used to grow, screen, and conduct experiments for different types of biological robots. The first level of the system 200 includes locating features and fixed stimuli, such as LEDs, electrical, vibration, and ambient light. The second level of the system 200 includes biological robot receptacles. The third level of the system 200 includes fluid handling and micro vision, such as x-y robot functionality, chemical, feeding, media changes, and micro-level vision cameras. The fourth level of the system 200 includes macro-level vision cameras. Each of these four levels is discussed in more detail below.

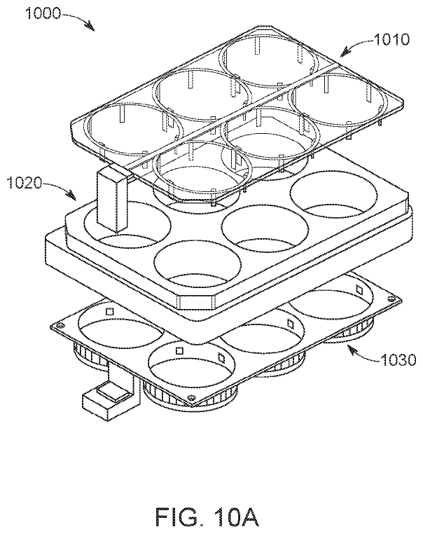

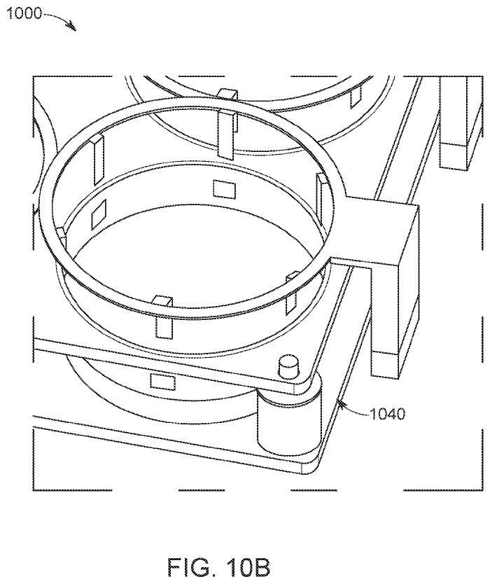

Referring to FIGS. 10A-10B, illustrations of an example biological robot receptacle tray 1000 that can be used with the system 200 are shown, in accordance with some aspects of the disclosure. The receptacle tray 1000 is a modular component that is representative of one possible receptacle configuration that can be used with the system 200. The receptacle tray 1000 is shown to include three parts: an electrode insert 1010, a well plate 1020, and a lighting insert 1030. The well plate 1020 is shown to include six separate wells that can each hold one or more biological robots. The well plate 1020 can hold six different 60-millimeter Petri dishes, for example. The lighting insert 1030 can be implemented as an LED ring that mounts on the bottom of the well plate 1020, for example. The lighting insert 1030 can also be implemented using various mechanisms for providing lighting stimuli (interventions) to biological robots maintained in the receptacle tray 1000.

The electrode insert 1010 can be secured to the top of well plate 1020, and can also be implemented using various mechanisms from providing stimuli (interventions) to biological robots. For example, the electrode insert 1010 can provide vibration stimuli to biological robots maintained in the receptacle tray 1000 via one or more mechanical transducers. The electrodes in the electrode insert 1010 can deliver electrical stimulus and/or acquire electrical data. The electrode insert 1010 and the lighting insert 1030 can accordingly be positioned to interact with the well plate 1020. FIG. 10B, specifically, shows an example vibration isolation mount 1040 formed between the electrode insert 1010, the well plate 1020, and the lighting insert 1030. This design can provide lighting that is arranged in a ring around each well of the well plate 1020, assemblies that snap in over the well plate 1020 that suspend electrodes in each well of the well plate 1020, plus isolation at the well plate level, in addition to modularity.

The first level of the system 200 as shown in FIG. 9 includes a set of modular inserts that can be reconfigured to support different functions. For example, the first level of the system 200 can include the electrode insert 1010 and the lighting insert 1030. The modular inserts can support different functions during the growth and experiments phases, and/or during the growth, screening, and experiments phases, such as discussed above. The different types of inserts can support different functions through simple location and orientation of well plates without stimuli support, simple location and orientation of Petri dishes without stimuli support, and/or fixed stimuli that “surround” the wells of the well plates. Nominally, in some examples, the first level has eight inserts that can each support two multi-well flat bottom well plates and four 60-millimeter Petri dishes. In some examples, fixed stimuli inserts are provided for at least two multi-well plate types and 60-millimeter Petri dishes. The first level can also use 6-well flat bottom well plates, 12-well flat bottom well plates, 24-well flat bottom well plates, among other sizes and types of well plates. The well plates are generally selected such that the width to depth ratio does not affect imaging of the edges of the wells. It will be appreciated the numerous different combinations of first level inserts can be implemented depending on the application.

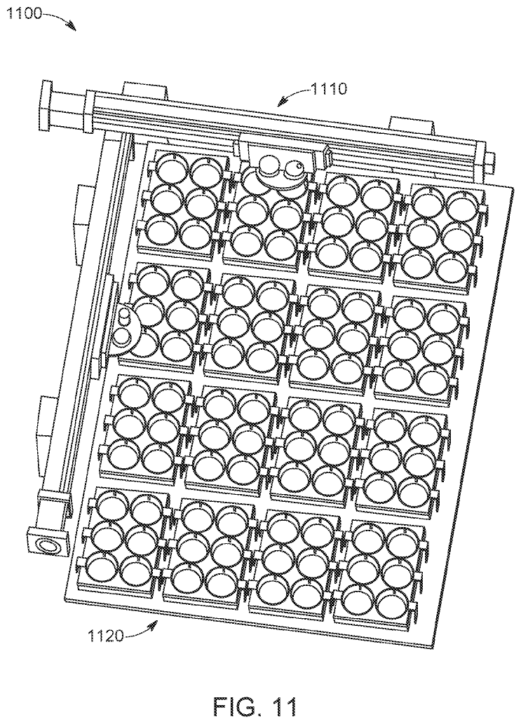

The second level of the system 200 as shown in FIG. 9 can be designed around two receptacle types: multi-well plates and 60-millimeter Petri dishes, in some examples. Stimuli can be delivered to any of the receptacle types during any portion of the process via the first level fixed stimuli inserts and the third level multi-function head, for example. Referring to FIG. 11, an illustration of an example biological robot receptacle control system 1100 is shown, in accordance with some aspects of the disclosure. The receptacle control system 1100 is shown to include a motion system 1110 and a plurality of receptacle trays 1120. Each receptacle tray of the plurality of receptacle trays 1120 can be the same as or similar to the example receptacle tray 1000, for example. The motion system 1100 can be a high precision x-y-z cartesian motion system that moves two multi-function heads between different positions relative to the plurality of receptacle trays 1120. The multi-function heads can be controlled by the multi-function head controller 260, for example, and can be used to perform dispensing actions 291 and extracting actions 292 of biological materials used to make and experiment with biological robots. In the example receptacle control system 1100 shown in FIG. 11, the top two rows of receptacle trays in the plurality of receptacle trays 1120 can be used during the growth phase, for example, while the bottom two rows of receptacle trays in the plurality of receptacle trays 1120 can be used during the experiments phase.

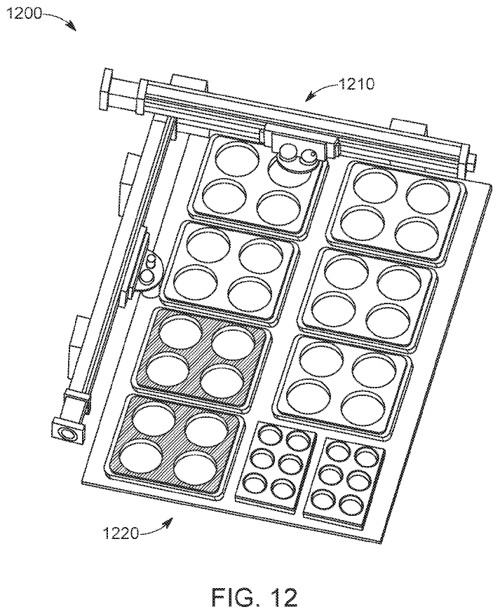

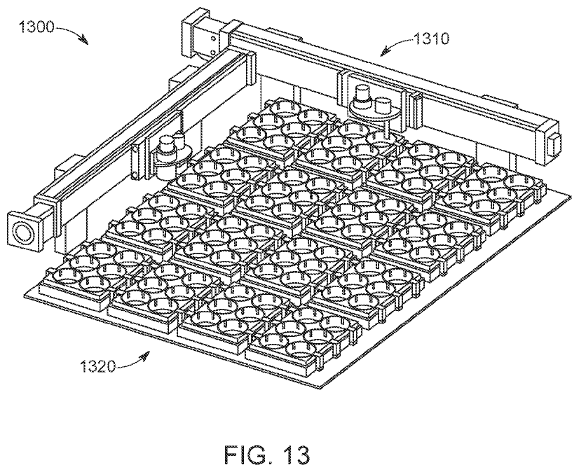

Referring to FIG. 12, an illustration of another example biological robot receptacle control system 1200 is shown, in accordance with some aspects of the disclosure. The receptacle control system 1200 is similar to the receptacle control system 1100 in that it includes a similar motion system 1210 and a similar plurality of receptacle trays 1220. However, the receptacle control system 1200 provides an example of another modular configuration that can be used with the system 200. In the receptacle control system 1200, both 4-well well plates and 6-well well plates are used. Also, the top two rows of receptacle trays in the plurality of receptacle trays 1220, and half of the third row of receptacle trays in the plurality of receptacle trays 1220, can be used for the experiments phase. Then, the other half of the third row of receptacle trays in the plurality of receptacle trays 1220 and half of the fourth row of receptacle trays in the plurality of receptacle trays 1220 can be used for the screening phase, and the other half of the fourth row of receptacle trays in the plurality of receptacle trays 1220 with the smaller sized wells can be used for the growth phase. FIG. 13 shows yet another example biological robot receptacle control system 1300, including a similar motion system 1310 and a similar plurality of receptacle trays 1320, that provides yet another example of a modular configuration that can be used with the system 200.

The third level of the system 200 as shown in FIG. 9 can include a multi-function head for liquid and biological robot handling via a pipette and a micro-level vision camera. For example, the third level can include the multi-function heads of the motion system 1110 and/or the motion system 1210. The liquid handling functionality can be used to change or add solution to different receptacles at different stages of biological robot growth, screening, and experiments phases. The micro-level vision camera can be used to capture detailed, close-up images of biological robots for various purposes. For example, the micro-level vision camera(s) may be capable of around 20× magnification with 0.81μ pixels, in some examples. The third level of the system 200 can also include a high precision x, y, z motion cartesian motion system (e.g., 3D cartesian robot, such as the motion system 1110 and/or the motion system 1210) that allows for the execution of all multi-function head capabilities at any location in the system. In some examples, the system 200 can include two robotic motion systems, such as two 3D cartesian robots. One of the cartesian robots may prod with a pipette, while the other views the biological robots with a micro-level camera, for example. Additional robots and different types of robots for performing these functions may be used depending on the application. Various types of fluid level detection systems and devices may also be used to monitor the level of fluids in the receptacles.

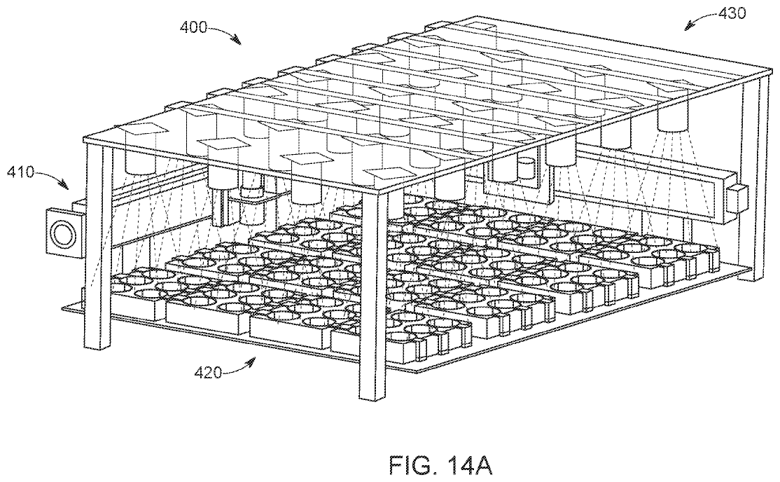

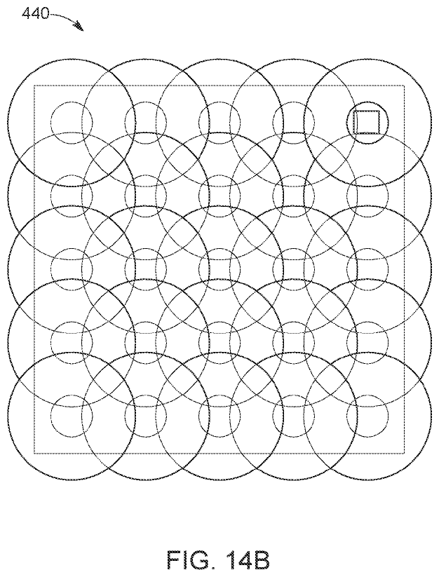

The fourth level of the system 200 as shown in FIG. 9 can include a suite of low-cost networked cameras (e.g., macro-level cameras in the observation camera system 206). The cameras can provide 100% field of view coverage for all receptacles and all biological robots in the system 200. A variety of different types of cameras can be used to implement this level of the system 200, and the cameras can be configured with programmable frame rates. Referring to FIG. 14A-14B, further illustrations of the monitoring system 400 are shown, in in accordance with some aspects of the disclosure. As shown in FIG. 14A, the monitoring system 400 includes a motion system 410 and a plurality of receptacle trays 420, similar to the motion system 1110 and the plurality of receptacle trays 1120 described above, for example. The monitoring system 400 also includes a plurality of cameras 430 that can be used to monitor biological robots contained within the plurality of receptacle trays 420. As shown in FIG. 14B, the coverage provided by the plurality of cameras 430 (e.g., camera array) is sufficient to cover the entirety of the plurality of receptacle trays 420. The plurality of cameras 430 can capture a variety of different types of images associated with biological robots, including infrared images and other types of images.

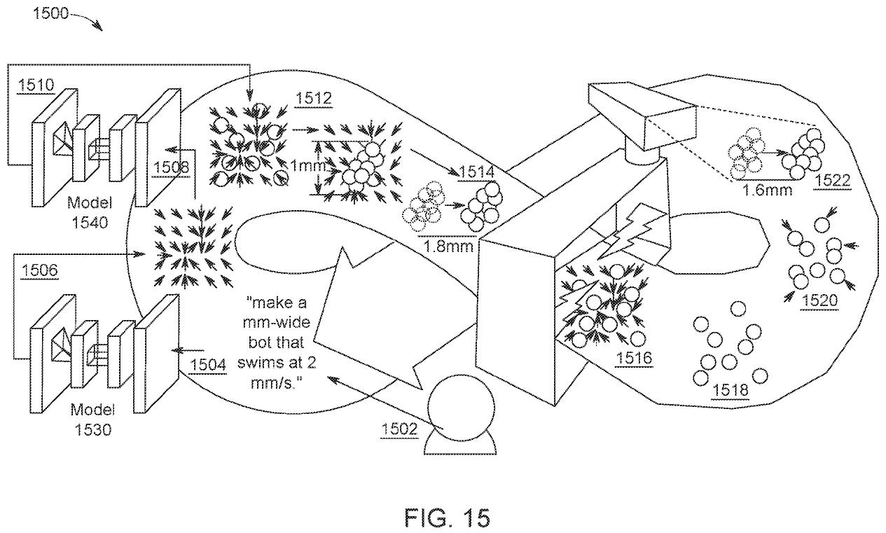

Referring to FIG. 15, an illustration of an example process 1500 for designing and making biological robots is shown, in accordance with some aspects of the disclosure. The process 1500 can be performed by the system 200, for example. The process 1500 generally involves generating and introducing interventions on designed biological robots using artificial intelligence methods. The artificial intelligence methods generally search space of all possible interventions and seeks those that, when provided to the biological robots, push the biological robots into a development trajectory that results in an adult organism that exhibits desired behavior.

As shown in FIG. 15, the process 1500 uses two artificial intelligence models: a first model 1530 and a second model 1540. It will be appreciated that the functionality of the first model 1530 and the second model 1540 can be integrated into a single model such that the first model 1530 and the second model 1540 are separate components of the same model. The functionality of the first model 1530 and the second model 1540 can also be integrated into separate models (e.g., two or more models) in some examples. Also, one or more models with different functionality and/or characteristics as the first model 1530 and the second model 1540 can be used to implement the system 200. The first model 1530 and the second model 1540 can be implemented using a variety of different types and combinations of machine learning and artificial intelligence algorithms, including different types of neural networks, support-vector machines, decision trees, linear and/or logistic regression models, clustering algorithms, and/or anomaly detection algorithms. The first model 1530 and the second model 1540 can be implemented using supervised approaches, unsupervised approaches, deep learning approaches, and other types of approaches to developing and training artificial intelligence models.

The first model 1530 can generally be conceptualized as a “request2intervention” model component that generates interventions for biological robots in response to requests for desired behavior submitted by humans. That is, the first model 1530 can receive a request for desired behavior of one or more biological robots as input from a human and generate as output one or more interventions for applying to the one or more biological robots to produce the desired behavior requested by the human. The second model 1540 can generally be conceptualized as a “intervention2phenotype” model component that learns patterns indicative of how different interventions applied to biological robots affect the biological robots. That is, the second model 1540 an receive an intervention as input and generate as output a prediction as to how that intervention, supplied by the system 200, will affect a biological robot under construction.

The second model 1540 can be trained based on historical intervention data generated using the system 200. Many random interventions (stimuli) can be generated and supplied to biological robots using the system 200, and data indicative of how biological robots respond to different interventions can be assembled into one or more training datasets used to train the second model 1540. Accordingly, the second model 1540 can be trained using a second training dataset, where each element of the second training dataset includes an intervention and a phenotype that caused the intervention. The second model 1540 can then be trained using the second training dataset by supplying the second model 1540 with the interventions included in the second training dataset and having the second model 1540 generate, as output, a simulation of cells that accurately reflects the developmental trajectory observed in the system 200 when the intervention was supplied to real cells. As a result of this training, the second model 1540 can predict what kind of organism will result from a given intervention.

The first model 1530 can be trained based on descriptions of different phenotypes produced by the system 200. The first model 1530 can be trained using a first training dataset, where each element of the first training dataset includes an organism (e.g., biological robot) and a description of the organism. The description can be a verbal description of the behavior of the organism, for example, among other possible types of descriptions. Some or all of the descriptions used to assemble the first training dataset can be provided by one or biologists and/or other personnel with subject matter expertise. The first model 1530 can then be trained using the first training dataset by supplying each description in turn as a text prompt to the first model 1530, for example, and having the first model 1530 generate, as output, an intervention. The intervention output by the first model 1530 can then be sent to the already-trained second model 1540, and the second model 1540 can generate as output a prediction in the form of a simulated organism and its behavior. Errors between the behaviors of the simulated organism and the physical organism can then be computed by the system 200 and propagated backward through the first model 1530 to improve the performance of the first model 1530. As a result of this training, the first model 1530 can receive as input (e.g., from a human) a description of a desired biological robot and generate as output an intervention that is likely to result in a biological robot that exhibits the desired characteristics identified in the input.

At 1502, a user (e.g., a human) can provide an input to the system 200 that describes a desired biological robot. The input can describe how the desired biological robot should look and/or how the biological robot should behave. The input (request) can be provided by the user to the system 200 in a variety of ways, such as through a user interface presented via the first control computer 220. The user can also provide inputs using a variety of different user devices (e.g., smartphones, tablets, laptops, wearable devices, etc.) that can ultimately be received by the system 200. The input can be provided by selecting one or more user interface elements (e.g., drop-down selections, etc.) as a voice command, a text command, or the like. For example, as illustrated in FIG. 15, the input can be a voice command or a text command that states “make a 1-millimeter-wide wide bot that swims at 2 millimeters per second” and is received by the system 200. In some instances, the input provided at 1502 may not necessarily be provided by a human.

At 1504, the input received from the user can be provided as input to the first model 1530. The first model 1530 can then process the input (e.g., using a deep neural network) to generate an intervention at 1506. Then, at 1508, the intervention can be provided as input to the second model 1540. The second model 1540 can then process the intervention to predict how the intervention will influence the behavior of a set of simulated dissociated cells at 1510 and predict a final phenotype that will result from the intervention at 1512. Data indicative of the differences between predicted phenotype and the desired phenotype can then drive changes in the first model 1530 and the second model 1540 during training (e.g., tuning weights of different nodes, layers, etc.).

At 1514, once the first model 1530 and the second model 1540 discover an intervention that matches the input received from the user, the intervention can be applied to real dissociated cells at 1516, thereby deflecting the cells' developmental trajectory (at 1518 and 1520) and causing them to form into an organism (biological robot) (at 1522) that exhibits the behavior requested by the user. Data indicative of the differences between the organism's observed behavior and the requested behavior can again be used to drive changes in the first model 1530 and the second model 1540 during training (e.g., tuning weights of different nodes, layers, etc.).

In the foregoing description, it will be readily apparent to one skilled in the art that varying substitutions and modifications may be made without departing from the scope and spirit of the disclosure. The disclosure suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that modification and/or variation of the concepts herein disclosed may be resorted to by those skilled in the art, and that such modifications and variations are considered to be within the scope of this disclosure.

1. A system for making biological robots, comprising:
a well plate comprising a receptacle;
a robotic assembly configured to form a biological robot in the receptacle by dispensing biological material into the receptacle;
an insert positioned to interact with the receptacle, the insert comprising one or more components configured to stimulate to the biological robot;
a camera system configured to monitor the biological robot in the receptacle; and
a computing system communicatively coupled to the robotic assembly and configured to:
receive an input from a user indicative of a desired behavior of the biological robot;
apply the user input as input to an artificial intelligence model;
identify a stimulus for providing to the biological robot such that the biological robot exhibits the desired behavior based on an output of the artificial intelligence model; and
cause the insert to provide the stimulus to the biological robot.

2. The system of claim 1, wherein the computing system is further configured to:
receive data from the camera system indicative of a response that is exhibited by the biological robot to the stimulus; and
use the data indicative of the response exhibited by the biological robot to the stimulus to train the artificial intelligence model.

3. The system of claim 1, wherein:
the artificial intelligence model comprises both a first model component and a second model component;
the first model component is configured to identify the stimulus for providing to the biological robot such that the biological robot exhibits the desired behavior based on the input from the user; and
the second model component is configured to identify a phenotype that will result from providing the stimulus to the biological robot.

4. The system of claim 1, wherein the artificial intelligence model comprises a deep learning neural network.

5. The system of claim 1, wherein the stimulus comprises at least one of a lighting stimulus, a vibration stimulus, an electrical stimulus, or a heating stimulus.

6. The system of claim 1, wherein the insert comprises at least one of a lighting insert including one or more light-emitting diode components or an electrode insert including one or more electrode components.

7. The system of claim 1, wherein the biological material comprises at least one of frog cells, human cells, nanomaterials, or deoxyribonucleic acid.

8. A method for making biological robots, comprising:
maintaining, by a computing system, an artificial intelligence model used for designing biological robots;
generating, by the computing system based on a first output of the artificial intelligence model, a first design for a biological robot;
causing, by the computing system, a robotic assembly to form the biological robot based on the first design in a first contained environment;
causing, by the computing system, a stimulus to be provided to the biological robot in the first contained environment;
receiving, by the computing system, data indicative of a response that is exhibited by the biological robot to the stimulus;
training, by the computing system, the artificial intelligence model using the data indicative of the response that is exhibited by the biological robot to the stimulus;
generating, by the by the computing system based on a second output of the artificial intelligence model, a second design for the biological robot; and
causing, by the computing system, the robotic assembly to form the biological robot based on the second design in a second contained environment.

9. The method of claim 8, wherein the first contained environment comprises a first Petri dish and the second contained environment comprises a second Petri dish.

10. The method of claim 8, wherein generating the first design for the biological robot based on the first output of the artificial intelligence model comprises applying a user input indicative of a desired behavior of the biological robot as input to the artificial intelligence model.

11. The method of claim 8, wherein causing the robotic assembly to form the biological robot based on the first design in the first contained environment comprises instructing the robotic assembly to dispense biological material in the first contained environment in accordance with the first design.

12. The method of claim 8, wherein causing the stimulus to be provided to the biological robot in the first contained environment comprises causing an insert that is positioned to interact with the first contained environment to provide a lighting stimulus, a vibration stimulus, an electrical stimulus, or a heating stimulus to the biological robot in the first contained environment.

13. The method of claim 8, wherein receiving the data indicative of the response that is exhibited by the biological robot to the stimulus comprises receiving the data indicative of the response that is exhibited by the biological robot to the stimulus from a camera system.

14. The method of claim 8, wherein the artificial intelligence model comprises a deep learning neural network.

15. A system for making biological robots, comprising:
a well plate comprising a plurality of receptacles;
a motion system configured to control a position of a head that that dispenses biological material into the plurality of receptacles to form biological robots in the plurality of receptacles;
an insert positioned to interact with the plurality of receptacles, the insert comprising one or more components configured to provide a stimulus to the biological robots;
a camera system disposed over the plurality of receptacles; and
a controller communicatively coupled to the insert, the camera system, and the motion system, the controller configured to cause the insert to provide the stimulus to the biological robots based on instruction from a computing system and to provide data from the camera system to the computing system.

16. The system of claim 15, wherein the biological material comprises at least one of frog cells, human cells, nanomaterials, or deoxyribonucleic acid.

17. The system of claim 15, wherein:
the insert comprises a first insert and the stimulus comprises a first stimulus;
the first insert is positioned across a top surface of the well plate;
the system further includes a second insert positioned across a bottom surface of the well plate, the second insert comprising one or more components configured to provide a second stimulus to the biological robots.

18. The system of claim 15, wherein the motion system comprises a cartesian motion system and the head comprises a multi-function head that dispenses the biological material into the plurality of receptacles from a pipette.

19. The system of claim 15, wherein:
the camera system comprises a camera array configured to generate infrared images of the biological robots; and
the stimulus comprises at least one of a lighting stimulus, a vibration stimulus, an electrical stimulus, or a heating stimulus.

20. The system of claim 15, wherein the controller is further configured to cause the motion system to form the biological robots in the plurality of receptacles based on instruction from the computing system.
